(19) United States
(12) Patent Application Publication
(10) Pub. No.: US 2023/0225651 A1
(43) Pub. Date: Jul. 20, 2023

(54) COGNITIVE FUNCTION TEST SERVER AND METHOD

(71) Applicant: LOOXID LABS INC., Daejeon (KR)

(72) Inventors: Yong Wook CHAE, Seoul (KR); Bo Hee LEE, Goyang-si Gyeonggi-do (KR); Jae Son JANG, Goyang-si Gyeonggi-do (KR); Tae Heon LEE, Seoul (KR); Song Sub LEE, Seoul (KR); Ki Bum KIM, Seoul (KR); Hyung Sin JEON, Siheung-si Gyeonggi-do (KR); Jeong Hwan HWANG, Seoul (KR)

(73) Assignee: LOOXID LABS INC., Daejeon (KR)

(21) Appl. No.: 17/778,854

(22) PCT Filed: Oct. 5, 2021
(86) PCT No.: PCT/KR2021/013628

§ 371 Date: May 22, 2022

(30) Foreign Application Priority Data
Oct. 8, 2020 (KR) 10-2020-0130496
Aug. 12, 2021 (KR) 10-2021-0106731

(51) Int. Cl. A61B 5/16; A61B 5/374; A61B 5/00

(52) U.S. Cl. CPC A61B 5/165; A61B 5/374; A61B 5/4088; A61B 5/6803

(57) ABSTRACT

The present invention relates to a cognitive function test server, including a communication interface, a memory; and a processor which is operably connected to the communication interface and the memory, and the processor is configured to provide a first sequence to acquire brainwave data of a user in a resting state by means of an HMD device, acquire baseline brainwave data of the user based on the first sequence, provide at least one second sequence related to a cognitive function by means of the HMD device, acquire input data and activated brainwave data based on the second sequence from the HMD device and an input device connected thereto, and generate a cognitive evaluation result of the user based on at least one of the reference brainwave data, the activated brainwave data, and the input data of the user.

### BACKGROUND OF THE DISCLOSURE

### Technical Field

The present invention relates to a server and a method for testing a cognitive function.

### Background Art

Recently, as it enters an aging society, a proportion of the elderly population continuously increases so that the interest in diseases, such as dementia, that are more likely to develop as people get older, is increasing.

Dementia refers to an acquired brain disease that the cognitive ability to remember and think gradually declines due to various causes to significantly interfere with the daily life.

Even though due to various and complex symptoms and causes of the dementia, there is no clear treatment for the dementia, but early diagnosis can delay the onset or alleviate the condition. Accordingly, for the purpose of the early diagnosis of the dementia, dementia diagnostic tests for the elderly are being actively conducted.

The background of the present invention is described for easier understanding of the present invention. It should not be understood to admit the matters described in the background of the present invention as a prior art.

### SUMMARY OF THE DISCLOSURE

The system of the related art has an advantage in that the cognitive function can be measured and trained using the HMD device anytime and anywhere. However, the system of the related art mostly evaluates the cognitive function based on a response speed and a response time for the stimulus so that the accuracy of the evaluation result is low.

Further, most of the systems of the related art are early studies using the HMD device, only the configuration of the connection between the HMD device and a cognitive ability test device is described. But studies about how to provide various types of VR contents to the HMD device and how to diagnose the cognitive ability based thereon have not been disclosed.

Accordingly, a method for accurately diagnosing a cognitive ability of a user by providing various VR-based contents for a cognitive ability test is being demanded.

As a result, the inventors of the present invention tried to develop a cognitive function testing method for providing a content sequence for testing a cognitive ability and diagnosing the cognitive ability of the user based on brainwave data and a server performing the method.

At this time, the inventors of the present invention configured a method for determining a cognitive evaluation result of a user by a combination of input data acquired from the HMD device and brainwave data or gaze data acquired from the user who performed the test.

Objects of the present invention are not limited to the above-mentioned objects, and other objects, which are not mentioned above, can be clearly understood by those skilled in the art from the following descriptions.

In order to achieve the above-described object, a cognitive function test server according to an exemplary embodiment of the present invention is provided. The server includes: a communication interface; a memory; and a processor which is operably connected to the communication interface and the memory, and the processor is configured to provide a first sequence to acquire brainwave data of a user in a resting state by means of an HMD device, acquire baseline brainwave data of the user based on the first sequence, provide at least one second sequence related to a cognitive function by means of the HMD device, acquire input data and activated brainwave data based on the second sequence from the HMD device and an input device connected thereto, and generate a cognitive evaluation result of the user based on at least one of the reference brainwave data, the activated brainwave data, and the input data of the user.

According to a feature of the present invention, the processor may be configured to provide a first sequence including at least one of a guide text configured to guide a user an eye-related action and an effect sound for guiding a specific time.

According to another feature of the present invention, the processor may be configured to provide an image for inducing the resting state.

According to still another feature of the present invention, the processor may be configured to calculate an evaluation score for every cognitive function of the user based on the input data, determine whether the user has an cognitive impairment according to the calculated evaluation score, and determine the effectiveness of the determined cognitive impairment based on a comparison result of the reference brainwave data and the activated brainwave data.

According to still another feature of the present invention, the processor may be configured to calculate a cognitive evaluation score of the user based on the input data, and determine whether the user has a cognitive impairment by reflecting a comparison result of the reference brainwave data and the activated brainwave data to the calculated evaluation score.

According to still another feature of the present invention, the processor may be configured to determine whether the user has a cognitive impairment based on a comparison result of the reference brainwave data and the reactive brainwave data, and provide the cognitive evaluation score calculated based on the input data as reference data.

According to still another feature of the present invention, the comparison result may be a power spectral density (PSD) comparison result for every frequency band.

According to still another feature of the present invention, whether the user has a cognitive impairment may be determined for every cognitive function of the user.

According to still another feature of the present invention, when the processor acquires a detaching signal of the HMD device, the processor may be configured to acquire the reference brainwave data of the user again by means of the HMD device.

According to still another feature of the present invention, the processor may be configured to measure a pupil movement of the user to compensate for a center of the pupil of the user or a distance between centers of the pupils.

According to still another feature of the present invention, the processor may be configured to provide a second sequence configured to test at least one cognitive function of short-term memory, long-term memory, concentration, depth perception, quickness, visual perception, spatial perception, working memory, instantaneous attention, sustained attention, reading ability, and selective attention of the user.

According to still another feature of the present invention, a second sequence configured to test the short-term memory or the long-term memory may include at least one of a plurality of graphic objects located in a three-dimensional space, a graphic object moving in the three-dimensional space, and a guide content guiding to selection of an object equal to or different form the plurality of graphic objects after a specific time, on the background of the three-dimensional space.

According to still another feature of the present invention, a second sequence configured to test the concentration may include at least one of a plurality of graphic objects having the same shape, at least one graphic object exposed in a different way after a predetermined time, among the plurality of graphic objects, a motion of the plurality of graphic objects in the three-dimensional space, and a guide content guiding to select at least one graphic object exposed in a different way.

According to still another feature of the present invention, a second sequence configured to test the depth perception may include at least one of a plurality of graphic objects having the same shape but located far or near from the user's point of view, and a guide content guiding to sequentially select graphic objects located far or near among the plurality of graphic objects.

In order to achieve the above-described object, a cognitive function testing method according to another exemplary embodiment of the present invention is provided. The method includes, providing a first sequence configured to acquire brainwave data of a user in a resting state by means of an HMD device; acquiring baseline brainwave data of the user based on the first sequence; providing at least one second sequence related to a cognitive function to the user in the resting state, by means of the HMD device; acquiring input data based on the second sequence from the HMD device and an input device connected thereto; and generating an evaluation result for every cognitive function of the user based on the reference brainwave data and the input data of the user.

Other detailed matters of the exemplary embodiments are included in the detailed description and the drawings.

According to the present invention, a brain state of a user for a series of sequences may be measured using the HMD device without a medical professional and the cognitive ability of the user may be evaluated based thereon.

Further, according to the present invention, a significant difference between a normal group and a patient group with a declined cognitive function is detected by means of brainwave data obtained from an EEG sensor mounted on the HMD device to diagnose an initial point of time when the cognitive function is declined. Specifically, a cognitive impairment is diagnosed at an early stage to prevent the sudden decline in the cognitive function.

Further, according to the present invention, reference brainwave data is measured again depending on whether the HMD device is detached, to increase the reliability of the test result.

The effects according to the present invention are not limited to the contents exemplified above, and more various effects are included in the present invention.

### BRIEF DESCRIPTION OF THE DRAWINGS

FIG. 1 is a schematic view of a cognitive function test system for supporting a cognitive function test according to an exemplary embodiment of the present invention.

FIG. 2 is a block diagram illustrating a cognitive function test server according to an exemplary embodiment of the present invention.

FIG. 3 is a flowchart of a cognitive function testing method according to an exemplary embodiment of the present invention.

FIGS. 4 to 15 are schematic views for explaining a sequence interface provided for a cognitive function test according to an exemplary embodiment of the present invention.

### DETAILED DESCRIPTION OF THE DISCLOSURE

Advantages and features of the present invention, and methods for accomplishing the same will be more clearly understood from exemplary embodiments described below with reference to the accompanying drawings. However, the present invention is not limited to the exemplary embodiment disclosed herein but will be implemented in various forms. The exemplary embodiments are provided by way of example only so that a person of ordinary skill in the art can fully understand the disclosures of the present invention and the scope of the present invention. Therefore, the present invention will be defined only by the scope of the appended claims. With regard to the description of drawings, like reference numerals denote like components.

In this specification, the terms “have”, “may have”, “include”, or “may include” represent the presence of the characteristic (for example, a numerical value, a function, an operation, or a component such as a part), but do not exclude the presence of additional characteristic.

In the specification, the terms “A or B”, “at least one of A or/and B”, or “at least one or more of A or/and B” may include all possible combinations of enumerated items. For example, the terms “A or B”, “at least one of A or/and B”, or “at least one or more of A or/and B” may refer to an example which includes (1) at least one A, (2) at least one B, or (3) all at least one A and at least one B.

Although the terms “first”, “second”, and the like, may be used herein to describe various components regardless of an order and/or importance, the components are not limited by these terms. These terms are only used to distinguish one component from another. For example, a first user device and a second user device may refer to different user devices regardless of the order or the importance. For example, without departing from the scope of the present invention, a first component may be referred to as a second component, and similarly, a second component may be referred to as a first component.

When a component (for example, a first component) is referred to as being “operatively or communicatively coupled with/to” or “connected to” another component (for example, a second component), it can be understood that the component is directly connected to the other element or connected to the other element via another component (for example, a third component). In contrast, when a component (for example, a first component) is referred to as being “directly coupled with/to” or “directly connected to” another component (for example, a second component), it is understood that there may not be another component (for example, a third component) between the components.

The terms “configured to (or set to)” may be exchangeably used with “suitable for”, “having the capacity to”, “designed to”, “adapted to”, “made to”, or “capable of” depending on the situation. The terms “configured to (or set)” may not necessarily mean only “specifically designed to” in a hardware manner. Instead, in some situations, the terms “a device configured to” may mean that the device is “capable of something” together with another device or components. For example, the terms “a processor configured (or set) to perform A, B, and C” may refer to a dedicated processor (for example, an embedded processor) configured to perform the corresponding operation or a generic-purpose processor (for example, a CPU or an application processor) which is capable of performing the operations by executing one or more software programs stored in a memory device.

The terms used in this specification are merely used to describe a specific exemplary embodiment, but do not intend to limit the scope of another exemplary embodiment. A singular form may include a plural form if there is no clearly opposite meaning in the context. Terms used herein including technical or scientific terms may have the same meaning as commonly understood by those skilled in the art. Among the terms used in this specification, terms defined in the general dictionary may be interpreted as having the same or similar meaning as the meaning in the context of the related art, but is not ideally or excessively interpreted to have formal meanings unless clearly defined in this specification. In some cases, even though the terms are defined in this specification, the terms are not interpreted to exclude the exemplary embodiments of the present specification.

The features of various exemplary embodiments of the present invention can be partially or entirely bonded to or combined with each other and can be interlocked and operated in technically various ways understood by those skilled in the art, and the exemplary embodiments can be carried out independently of or in association with each other.

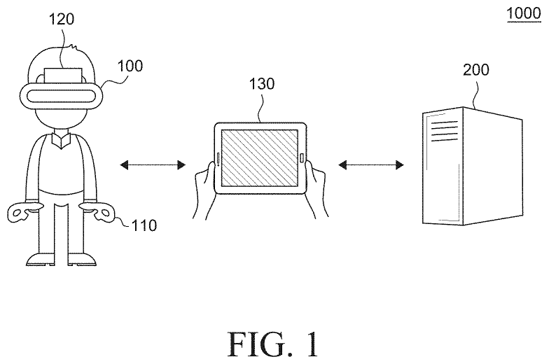

FIG. 1 is a schematic view of a cognitive function test system for supporting a cognitive function test according to an exemplary embodiment of the present invention.

Referring to FIG. 1, the cognitive function test system 1000 is a test system that tests a cognitive function and determines whether a user has a cognitive impairment. The cognitive function test system 1000 may include an HMD device 100 mounted by the user and a cognitive function test server 200 that tests the user's cognitive function and determines the cognitive impairment. Here, the cognitive impairment may include dementia such as mild cognitive impairment, Alzheimer, and Parkinson's disease, pseudodementia such as depression, and the like.

The HMD device 100 may be mounted on a head of the user and provide the user with spatial and temporal experience similar to the reality. The HMD device 100 may be a complex virtual experience device capable of sensing physical, cognitive, and emotional changes of the user who is undergoing a virtual experience.

The HMD device 100 may receive and output a sequence for testing the cognitive function, at the same time, acquire biometric data such as gaze or brain wave of the user. To this end, an input device 110 and at least one sensor 120 may be connected to the HMD device 100.

In the meantime, the sequence may be understood as various types of contents that can be output through the HMD device 100 and an output device connected thereto. The sequence is cognized by the user, such as images, audios, videos, vibrations, and motions.

The input device 110 may acquire input data by a left hand and a right hand of the user. Specifically, the input device 110 may acquire the input data in response to a stimulus displayed on the HMD device 100.

The sensor 120 may include at least one of a gaze sensor measuring a user's gaze and a brainwave sensor measuring a brain wave. For example, the gaze sensor may be a camera module that photographs an image including a pupil and a brainwave sensor may be an electroencephalogram (EEG) sensor. In the meantime, the brainwave sensor used for the cognitive function test is not disposed in the entire head of the user, but the brainwave sensor may be disposed in a part of a frontal lobe of the user. However, the brainwave sensor is not limited thereto and the brainwave sensor may include a functional magnetic resonance imaging (fMRI), a near infrared spectroscopy (fNIRS), or the like.

The HMD device 100 may be implemented to process virtual reality, augmented reality, and mixed reality (VR, AR, and MR) images in the device. Or a separate display device (not illustrated) providing the above-mentioned image is mounted in a part of the HMD device 100 to process the image therethrough. For example, the HMD device 100 may receive and output a sequence for a cognitive function test. Further, the HMD device 100 may transmit various data acquired with regard to the output sequence to a manager device 130 or a cognitive function test server 200.

According to the exemplary embodiment, the HMD device 100 may provide a sequence for testing a cognitive function of the user such as short-term memory, long-term memory, reading ability, concentration, depth perception, and quickness of the user. The HMD device 100 may acquire input data and biometric data (for example, gaze data or brainwave data) based on a sequence and output a cognitive function evaluation result determined based on the input data and the biometric data from the cognitive function test server 200.

In the meantime, when the user detaches the HMD device 100, the HMD device 100 may transmit a detaching signal of the HMD device 100 of the user to the cognitive function test server 200. When the user wants to proceed the cognitive function test after the detaching signal, the HMD device 100 may receive and output a sequence for acquiring user biometric data in a resting state from the cognitive function test server 200.

The manager device 130 may provide a user interface for supporting the cognitive function test of the user. The manager device 130 may be implemented by a smart phone, a tablet PC, or a PC capable of outputting an interface screen.

The manager device 130 is connected to the HMD device 100 to exchange various data for a cognitive function test. The manager device 130 shares a screen of the HMD device 100 worn on the user to support the HMD device 100 with device connection, communication connection, a tutorial progress, a cognitive function test, and an analysis result.

The cognitive function test server 200 may perform the cognitive function test and generate a cognitive evaluation result. The cognitive function test server 200 may be implemented by a general-purpose computer, a laptop computer, a cloud server, and the like.

According to the exemplary embodiment, the cognitive function test server 200 may receive input data and biometric data from the HMD device 100 or the manager device 130. The cognitive function test server 200 may generate an evaluation result for the cognitive function test based on the received input data and biometric data. Thereafter, the cognitive function test server 200 may provide the generated evaluation result data to the HMD device 100 or the manager device 130.

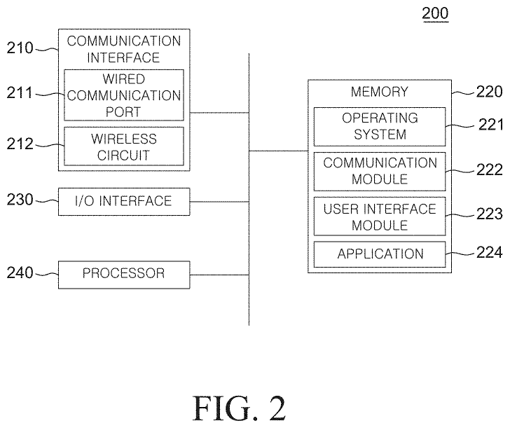

Hereinafter, a cognitive function test server 200 which tests the cognitive function will be described in more detail with reference to FIG. 2.

FIG. 2 is a block diagram illustrating a cognitive function test server according to an exemplary embodiment of the present invention.

Referring to FIG. 2, the cognitive function test server 200 may include a communication interface 210, a memory 220, an I/O interface 230, and a processor 240 and each configuration may communicate with each other via one or more communication buses or signal lines.

The communication interface 210 is connected to the HMD device 100 and the manager device 130 via a wired/wireless communication network to exchange data. For example, the communication interface 210 may provide a sequence to the HMD device 100 and the communication interface 210 may receive input data and biometric data (gaze data and brainwave data) based on the sequence from the HMD device 100. As another example, the communication interface 210 may receive user data who performs the cognitive function test from the manager device 130. Further, the communication interface 210 may transmit a cognitive function test history, a recovery history, a diagnosis result, and the like of the user to the manager device 130.

In the meantime, the communication interface 210 which allows the data to be transmitted/received may include a communication port 211 and a wireless circuit 212. Here, the wired communication port 211 may include one or more wired interfaces, for example, Ethernet, universal serial bus (USB), Firewire, or the like. Further, the wireless circuit 212 may transmit and receive data with external devices by an RF signal or an optical signal. In addition, the wireless communication may use at least one of a plurality of communication standards, protocols, and technologies, such as GSM, EDGE, CDMA, TDMA, Bluetooth, Wi-Fi, VoIP, Wi-Max, or other arbitrary appropriate communication protocols.

The memory 220 may store various data used in the cognitive function test server 200. For example, the memory 220 may store a sequence configured to cause the user to mediate and a sequence configured to test short-term memory, long-term memory, concentration, depth perception, quickness, visual perception, spatial perception, working memory, instantaneous attention, sustained attention, reading ability, and selective attention of the user. As another example, the memory 220 may store a cognitive evaluation score reference value for diagnosing a comprehensive cognitive impairment and cognitive impairment for every cognitive function, a brainwave data (for example, an EEG signal) difference between the normal group and a patient group with a declined cognitive function, and a pattern.

In various exemplary embodiments, the memory 220 may include a volatile or nonvolatile recording medium which may store various data, commands, and information. For example, the memory 220 may include at least one type of storing media of a flash memory type, a hard disk type, a multimedia card micro type, a card type memory (for example, an SD or XD memory), a RAM, an SRAM, a ROM, an EEPROM, a PROM, a network storage, a cloud, and a block chain database.

In various exemplary embodiments, the memory 220 may store a configuration of at least one of an operating system 221, a communication module 222, a user interface module 223, and one or more applications 224.

The operating system 221 (for example, an embedded operating system such as LINUX, UNIX, MAC OS, WINDOWS, VxWorks) may include various software components and drivers which control and manage a general system task (for example, memory management, storage device control, power management, or the like) and support communication between various hardware, firmware, and software components.

The communication module 222 may support communication with other devices through the communication interface 210. The communication module 222 may include various software components for processing data received by a wired communication port 211 or a wireless circuit 212 of the communication interface 210.

The user interface module 223 may receive a request or an input of the user from a keyboard, a touch screen, or a microphone via the I/O interface 230 and provide the user interface on the display.

The application 224 may include a program or a module configured to be executed by one or more processors 240. Here, the application for generating a cognitive evaluation result may be implemented on a server farm.

The I/O interface 230 may connect at least one of the input/output devices (not illustrated) of the cognitive function test server 200, such as a display, a keyboard, a touch screen, and a microphone, to the user interface module 223. The I/O interface 230 may receive the user input (for example, voice input, keyboard input, touch input, or the like) together with the user interface module 223 and process a command in accordance with the received input.

The processor 240 is connected to the communication interface 210, the memory 220, and the I/O interface 230 to control an overall operation of the cognitive function test server 200. The processor 240 may perform various commands to determine whether the user has a cognitive impairment by the application or the program stored in the memory 220.

The processor 240 may correspond to an arithmetic device such as a central processing unit (CPU) or an application processor (AP). Further, the processor 240 may be implemented as an integrated chip (IC) such as a system of chip (SoC) in which various arithmetic devices are integrated. Further, the processor 240 may include a module for calculating an artificial neural network model like a neural processing unit (NPU).

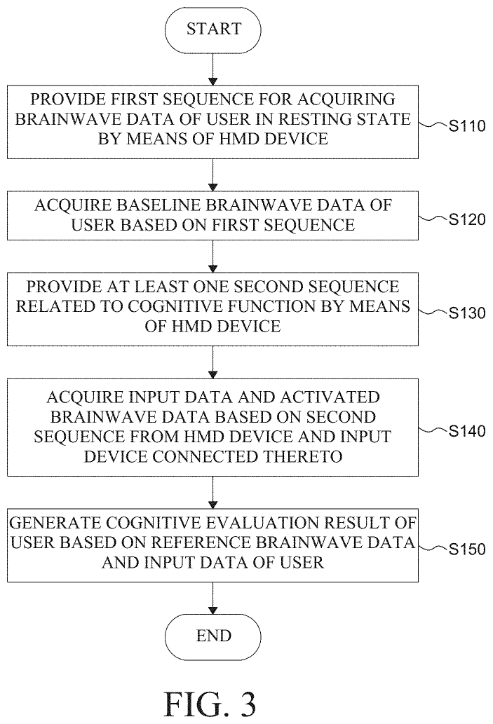

Hereinafter, a method for testing a cognitive function by a processor 240 of the cognitive function test server 200 will be described with reference to FIGS. 3 to

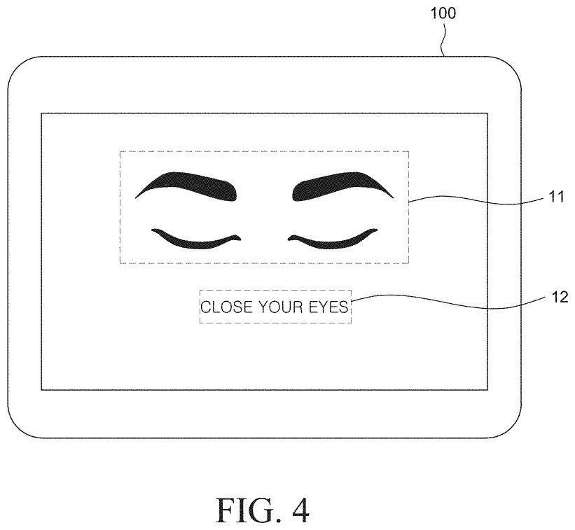

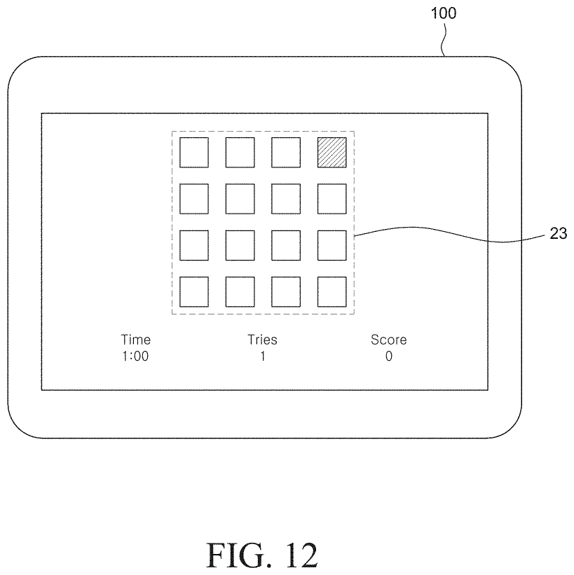

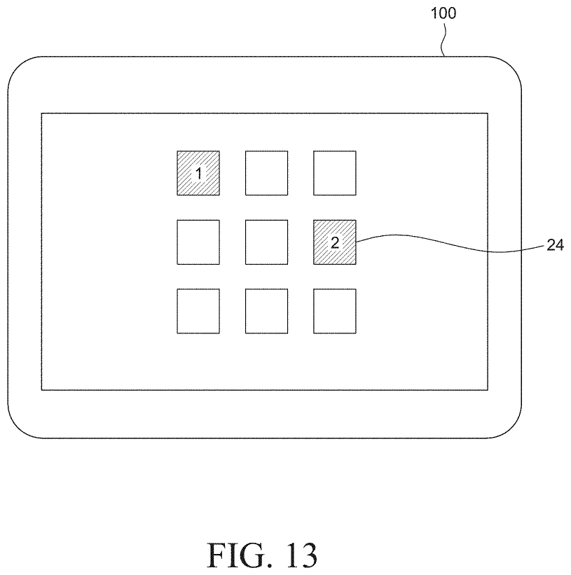

FIG. 3 is a flowchart of a cognitive function testing method according to an exemplary embodiment of the present invention. FIGS. 4 to 15 are schematic views for explaining a sequence interface provided for a cognitive function test according to an exemplary embodiment of the present invention.

Referring to FIG. 3, the processor 240 may provide a first sequence for acquiring user brainwave data in a resting state by means of the HMD device 100. Here, the first sequence is to put the user in a resting state and may help the user to mediate. Specifically, the first sequence may include at least one of a guide text configured to guide a user an eye-related action and an effect sound for guiding a specific time. At this time, the effect sound for guiding a specific time may be an effect sound for guiding an ending time of the mediation after completing the acquisition of a biometric data reference value of the user.

Referring to FIG. 4, the processor 240 may provide the following first sequence to the HMD device 100. Specifically, the first sequence may include an eye-related graphic object 11 or a guide text 12 such as “Close your eyes”. Further, the first sequence may include an audio-type guide text to help the mediation of the user. For example, the audio-type guide text may include a script such as “Relieve tension in your body in a comfortable position.”, “Breath in slowly.”, “Breath out slowly.”, “Breath in and out.”, and “Focus on the stillness between breaths and put your mind at ease.” and the given sentences may be provided to the user at a predetermined time interval.

According to the exemplary embodiment, the processor 240 may provide an image to induce the user in a resting state to the HMD device 100. For example, the image may be an image including natural scenes such as forests or seas.

After the step S110, the processor 240 may acquire baseline brainwave data of the user based on the first sequence (S120). Here, the reference brainwave data may be acquired by a sensor 120 included in the HMD device 100. The processor 240 may utilize the currently acquired reference brainwave data as comparison data with activated brainwave data acquired immediately later.

According to various exemplary embodiments, the first sequence may be any type of sequence as long as it is for creating a rest state, and the first sequence may include an interface capable of inducing a rest in the game, and the like. In other words, all the sequences that capable of acquiring the reference brainwave data are referred to as the first sequence and are intended not to be limited to a specific sequence.

Unlike an offline test that the manager directly observes the state of the user and stops or continues the test, in the case of the cognitive function test by means of the HMD device 100, there may be a situation that the user may take off the HMD device 100 during the cognitive function test. Thereafter, if the same user resumes the cognitive function test in the same condition, there is no problem. However, when another user mounts the HMD device 100 or the cognitive function test is performed in a different state, an accurate cognitive function test result of the corresponding user cannot be obtained.

Accordingly, when the processor 240 acquires the detaching signal of the HMD device 100, the processor 240 may acquire the reference brainwave data of the user through the HMD device 100 again. Specifically, the processor 240 may transmit a reacquired signal of the reference brainwave data to the HMD device 100.

According to the exemplary embodiment, the processor 240 measures a pupil movement of the user to compensate for a center of the pupil of the user or a distance between the centers of the pupils. That is, the processor 240 may adjust the center of the gaze of the user by means of eye tracking. The processor 240 may provide the sequence therefor through the HMD device 100. For example, the processor 240 may provide a guide text “Look at a dot on the center of the screen.” by means of the HMD device 100 to compensate for the center of the pupil of the user and provide a flickering red dot on the center of white, gray, and black backgrounds. As another example, the processor 240 may provide a guide text such as “Do not move your head and follow the dot only with your eyes.” by means of the HMD device 100 to compensate for the distance between the centers of the pupils. The processor 240 may provide a graphic object such as a gray dot or an arrow moving along a vertical direction (↑↓), diagonal directions [symbol] and a horizontal direction [symbol] at 40° interval with respect to the center point.

After the step S120, the processor 240 may provide at least one second sequence related to the cognitive function by means of the HMD device 100 (S130). Specifically, the processor 240 may provide the second sequence configured to test at least one cognitive function of short-term memory, long-term memory, concentration, depth perception, quickness, visual perception, spatial perception, working memory, instantaneous attention, sustained attention, reading ability, and selective attention of the user.

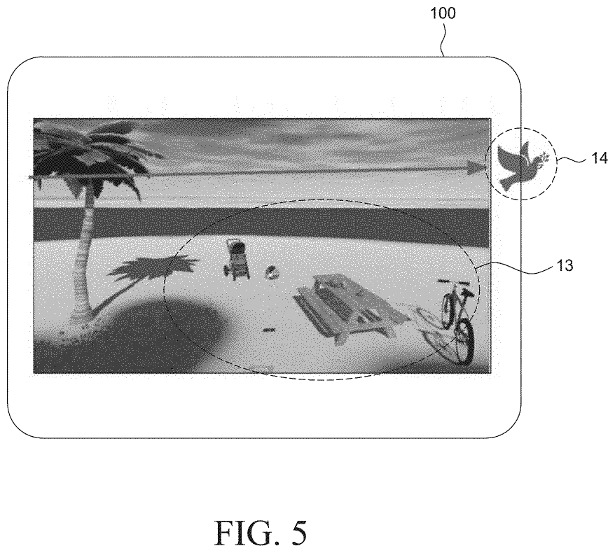

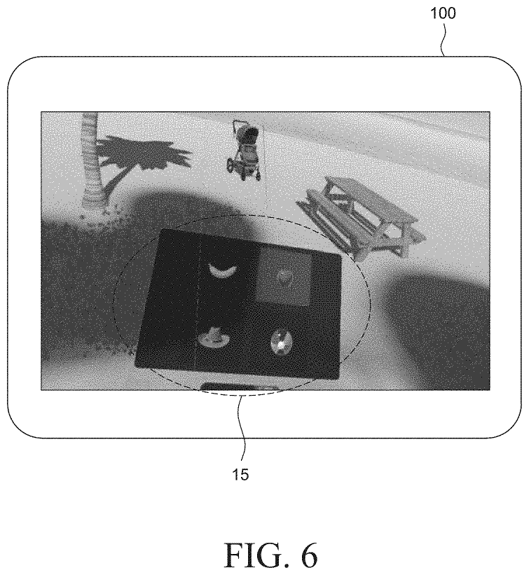

Referring to FIGS. 5 and 6, the processor 240 may provide a second sequence configured to test the short-term or long-term memory to the HMD device 100 as follows. Specifically, the second sequence is set in a three-dimensional space. Further, the second sequence may include at least one of a plurality of graphic objects 13 located in a three-dimensional space, a graphic object 14 moving in the three-dimensional space, and a guide content 15 guiding selection of an object equal to or different from the plurality of graphic objects 13 after a predetermined time.

Here, in the case of the second sequence configured to test the short-term memory, the processor 240, for example, may provide a guide text such as “Please remember positions of the objects on the beach.” to the HMD device 100. The processor 240 may provide a plurality of graphs objects 13 located in the three-dimensional space. The processor 240 may provide a guide text such as “Please choose the objects that were in the box a while ago and put them back in place.” to the HMD device 100 after a predetermined time (for example, 10 seconds).

In contrast, the second sequence configured to test the long-term memory the timing of providing a guide text may be changed. For example, after providing the plurality of graphic objects 13 located in the three-dimensional space, the processor 240 may provide a guide text such as “There are objects that you have not seen before on the beach. Please choose three objects that you have not seen.” to the HMD device 100 at a timing when at least one another second sequence is completed or at least one minute later.

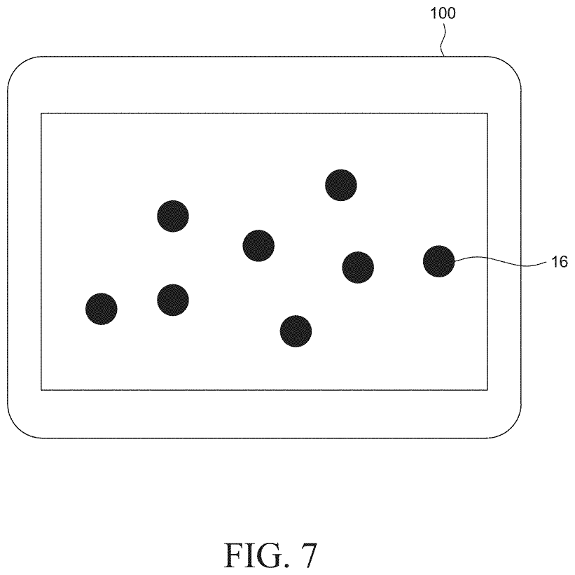

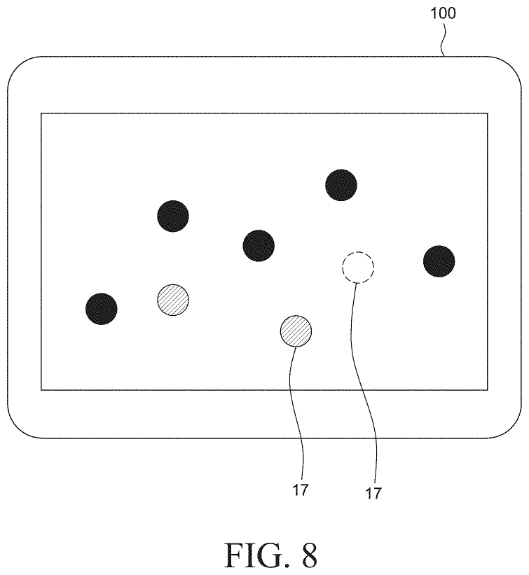

Referring to FIGS. 7 to 9, the processor 240 may provide a second sequence configured to test the concentration to the HMD device 100 as follows. Here, the backgrounds of FIGS. 7 and 8 may be implemented in white for the convenience of description. The background of the second sequence configured to test the attention may be implemented in black to further increase attention and concentration of the user.

The second sequence is set in a three-dimensional space. Further, the second sequence may include at least one of a plurality of graphic objects 16 having the same shape, a three-dimensional motion of the plurality of graphic objects 16, and a guide content guiding to select at least one graphic object 17 exposed in a different way among the plurality of graphic objects 16.

The processor 240 may provide a guide text about how to proceed the second sequence while providing the plurality of graphic objects 16 by means of the HMD device 100. For example, the processor 240 may provide a guide text such as “This is a game to remember the flickered ball.” and “Please remember the flickering ball well.” through the HMD device 100. Thereafter, the processor 240 may provide the plurality of graphic objects 16 having the same shape again. Further, the processor 240 may provide a motion that the plurality of graphic objects 16 repels each other on the screen of the HMD device 100 without overlapping. For example, when a distance between two graphic objects 16 is smaller than a predetermined value, the processor 240 may provide a motion to repel in a random direction. For example, after providing the motion of the plurality of graphic objects 16, the processor 240 may provide a guide text such as “Balls have stop. Can you find a ball that flickered at first?”.

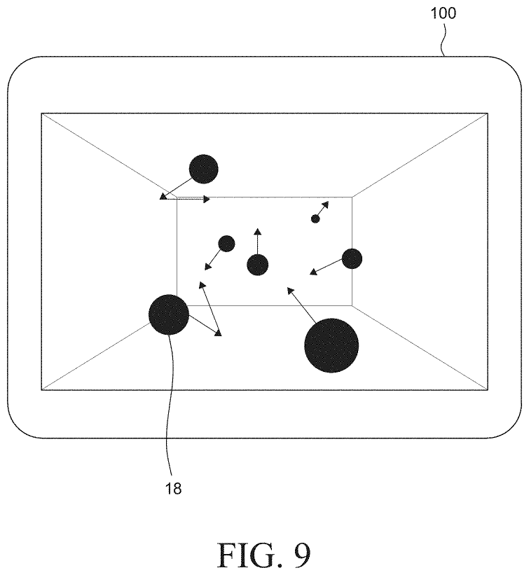

According to the exemplary embodiment, in the second sequence configured to test the concentration, the processor 240 may place the plurality of graphic objects 16 on a three-dimensional space as illustrated in FIG. 9, rather than a two-dimensional plane. In this case, the plurality of graphic objects 18 may be output such that a graphic object located to be far from the gaze of the user is small and a graphic object located to be close to the gaze is large. For example, the processor 240 may provide a guide text such as “Let's do it now in a wider space.”. the processor 240 may provide a motion that the plurality of graphic objects 18 repels each other in the screen of the HMD device 100 without overlapping. For example, when a distance between two graphic objects 18 is smaller than a predetermined value, the processor 240 may provide a motion and a graphic effect that the graphic objects repel in a random direction and the sizes become larger or smaller depending on the direction.

According to another exemplary embodiment, the processor 240 may provide a second sequence configured to test the concentration a plurality of times and the difficulty of the text may be increased at every try. For example, the second sequence configured to test the concentration is provided nine times, in first to third tries, one graphic object is exposed in a different way, among the plurality of graphic objects 16, in fourth to sixth tries, two graphic objects are exposed in a different way, and in seventh to ninth tries, three graphic objects are exposed in a different way.

Further, the processor 240 may select and provide a difficulty level of the second sequence configured to test the cognitive function by means of the HMD device 100 or the manager device 130.

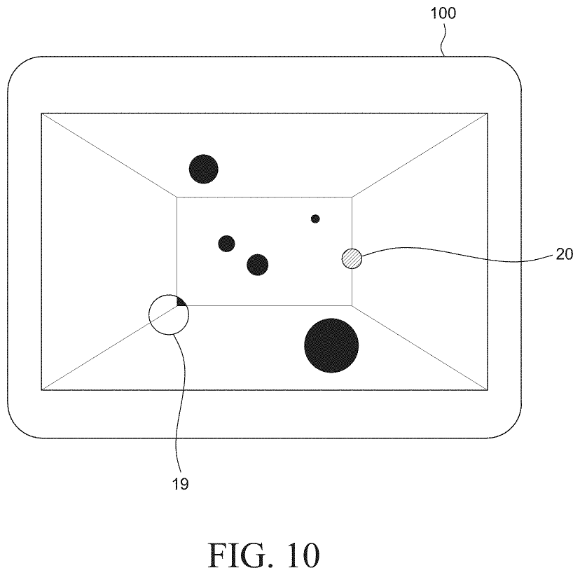

Referring to FIG. 10, a second sequence configured to test the depth perception may be provided to the HMD device 100 as follows. Specifically, the second sequence is set in a three-dimensional space. Further, the second sequence may include at least one of a plurality of graphic objects 19 having the same shape but located far or near from the user's point of view and a guide content guiding to sequentially select graphic objects 20 located far or near among the plurality of graphic objects 19.

The processor 240 may provide a guide text about how to proceed the second sequence while providing the plurality of graphic objects 20 located in different depths by means of the HMD device 100. For example, the processor 240 may provide a guide text such as “This game is to choose the balls from the nearest ball to the farthest ball in order.” and “Now choose the balls from the farthest ball to the nearest ball in order.” by means of the HMD device 100.

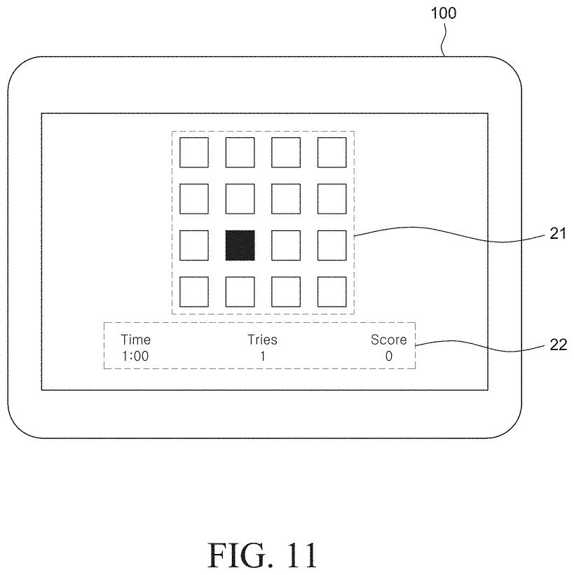

Referring to FIGS. 11 to 13, the processor 240 may provide a second sequence configured to test at least one of instantaneous attention, visual perception, and working memory. Specifically, the second sequence may include a graphic object 21 having the same shape and color, or a progress information text 22 including at least one of a second sequence running time, the number of tries, and a score. For example, the processor 240 may provide a guide text such as “This is a game to remember the order.” and “Some boxes light up in order. Please remember the order and the location of the lighted box.” through the HMD device 100. Thereafter, the processor 240 may provide sequentially one or more graphic objects 23 visually having different colors among the graphic objects having the same shape and color, by means of the HMD device 100. Further, the processor 240 may display a number 24 on the graphic object according to the difficulty. The processor 240 may provide a guide text such as “All the boxes that were lit up are turned off. Please choose the boxes that have just been lit up in the order you saw them.” by means of the HMD device 100.

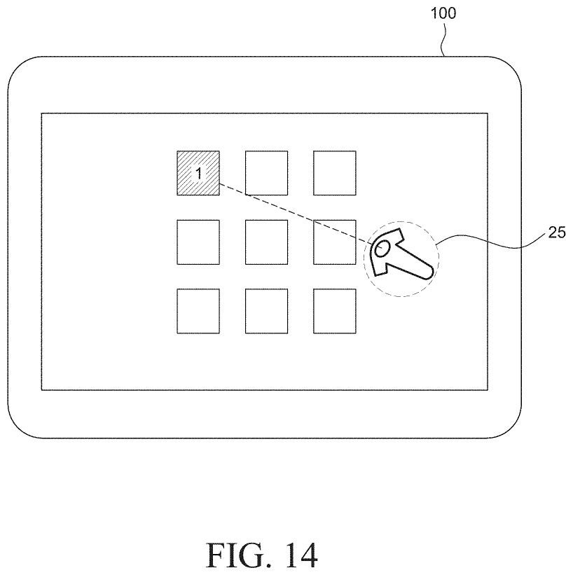

Furthermore, referring to FIG. 14, the processor 240 may provide a graphic object 25 for the input device 110 held by the user while the second sequence is provided by the HMD device 100.

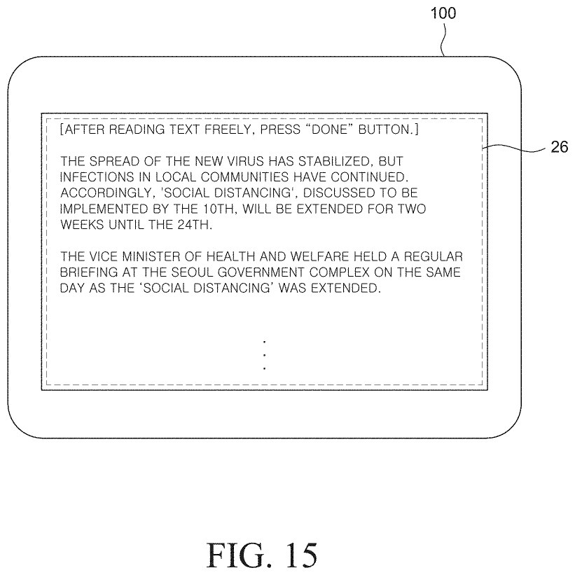

Referring to FIG. 15, the processor 240 may provide a second sequence configured to test reading ability or sustained attention. Specifically, the second sequence may include texts stored in the memory 220 regardless of the field. For example, the processor 240 may provide a guide text such as “After reading the text freely, click “Done” button.” by means of the HMD device 100. Further, the processor 240 may provide a text 26 arbitrarily selected from among the texts stored in the memory 220.

Referring to FIG. 3 again, after providing various types of second sequences as described above, the processor 240 may acquire input data and activated brainwave data based on the second sequence from the HMD device 100 and the input device connected thereto. Specifically, the processor 240 may acquire at least one input data, among the gaze data of the user, a response speed and the response time of the user according to the second sequence from the HMD device 100. The processor 240 may acquire at least one brain wave among an EGG signal, an EGG pattern, and a power (density) for every frequency band, by means of the sensor 120 connected to the HMD device 100.

After the step S140, the processor 240 may generate a cognitive evaluation result of the user based on at least one of the reference brainwave data, the activated brainwave data, and the input data of the user (S150). Specifically, the processor 240 may calculate a cognitive evaluation score of the user for the second sequence based on the input data. The processor 240 may generate a cognitive evaluation result of the user by a combination of the calculated cognitive evaluation score and the brainwave data. Further, the processor 240 may transmit the cognitive evaluation result of the user to the HMD device 100 or the manager device 130 by means of the communication interface 210.

According to the exemplary embodiment, the processor 240 may calculate the cognitive evaluation score of the user based on at least one of the user's response speed, response time, and score for the second sequence. The processor 240 may primarily determine whether the user has an impairment, according to the calculated evaluation score. Further, the processor 240 compares the reference brainwave data previously acquired in step S120 with the activated brainwave data acquired in step S140 to determine the effectiveness of the cognitive impairment of the user.

The processor 240 may acquire a comparison result of the brainwave data by the comparison of power spectral density (PSD) for every frequency band. Specifically, the processor 240 may obtain a difference between a PSD value of the reference brainwave data and a PSD value of the activated brainwave data in each of a delta band of 1 to 3 Hz, a theta band of 4 to 7 Hz, an alpha band of 8 to 14 Hz, a beta band of 15 to 30 Hz. That is, the processor 240 compares the difference in the PSD value of the user and the difference in the PSD value acquired from the normal group to determine whether the user has a cognitive impairment. For example, in the case of the depth perception, a PSD difference value of a patient with the cognitive impairment in the alpha band may be lower than the PSD difference value of the normal group.

In addition, the processor 240 may calculate a PSD value for every frequency band and utilize the ratio for every band as a comparison result.

Specifically, the processor 240 may calculate a delta/alpha ratio (DAR) or a theta/alpha ratio (TAR) and then compare the ratio of the normal group and a ratio calculated for the current user.

That is, when the primary cognitive impairment result determined using the input data matches a comparison result of the brainwave data, the processor 240 may determine that the primarily determined result on whether the user has a cognitive impairment is effective. If the primary cognitive impairment result and the comparison result of the brainwave data do not match, the processor 240 may correct the primarily determined result on whether the user has a cognitive impairment. Otherwise, the processor 240 may generate both the complicating results on whether the user has a cognitive impairment as a result. Further, the processor 240 may provide the cognitive evaluation score and the comment according to the comparison result of the brainwave data. The processor 240 may provide a comment for each section of a cognitive evaluation score and for each section of the difference values of the brainwave data. The comment may be a comment input by the manager or a medical staff to be stored in the memory 220.

According to another exemplary embodiment, the processor 240 may calculate the cognitive evaluation score of the user based on at least one of the response speed, the response time, and the score of the user for the second sequence. The processor 240 reflects the comparison result of the reference brainwave data and the activated brainwave data to the calculated evaluation score to determine whether the user has a cognitive impairment. That is, unlike the previous exemplary embodiment which primarily determines whether the user has a cognitive impairment and verifies the validity, the processor 240 may generate one diagnosis result by complementing the results acquired from two types of data.

According to another exemplary embodiment, the processor 240 may determine whether the user has a cognitive impairment based on the comparison result of the reference brainwave data and reactive brainwave data. The processor 240 may provide the cognitive evaluation score calculated based on the input data to the HMD device 100 or the manager device 130 as reference data. That is, in the present exemplary embodiment, the cognitive brainwave data may be preferentially considered to generate the cognitive evaluation result.

In the meantime, in the above-described two exemplary embodiments, the comparison result of the brainwave data may be a PSD comparison result for every frequency band.

As described above, the processor 240 may generate a cognitive evaluation result by means of various methods. The previous cognitive evaluation result is a result obtained by combining several types of cognitive functions and if necessary, the processor 240 may generate a cognitive evaluation result for every cognitive function. Specifically, every cognitive function may include short-term memory, long-term memory, concentration, depth perception, quickness, visual perception, spatial perception, working memory, instantaneous attention, sustained attention, reading ability, and selective attention of the user.

Until now, the cognitive function test server 200 according to the exemplary embodiment of the present invention has been described. According to the present invention, unlike the related art that roughly diagnoses the cognitive ability by means of the HMD device 100, the cognitive function test server 200 performs the diagnosis by reflecting the brainwave data. Thereafter, the user may perform the professional diagnosis by him/herself and also diagnose the initial cognitive impairment by means of the HMD device 100. Further, the cognitive function test server 200 measures the reference brainwave data again depending on whether the HMD device 100 is detached to enhance the reliability of the test result.

In the meantime, the method of providing a series of sequences and testing a cognitive function which is performed in the cognitive function test server 200 may also be performed in the HMD device 100. In this case, the HMD device 100 may store a sequence configured to cause the user to mediate and a sequence configured to test short-term memory, long-term memory, concentration, depth perception, quickness, visual perception, spatial perception, working memory, instantaneous attention, sustained attention, reading ability, and selective attention of the user. As another example, the HMD device 100 may store a cognitive evaluation score reference value for diagnosing a comprehensive cognitive impairment and cognitive impairment for every cognitive function, a brainwave data (for example, an EEG signal) difference between the normal group and a patient group with a declined cognitive function, and a pattern. The HMD device 100 may calculate the cognitive evaluation score of the user based thereon, and determine the effectiveness on whether the user has a cognitive impairment by comparing the calculated score and the brainwave data. Further, all the results performed by the HMD device 100 may be displayed on a display screen of the HMD device 100 or a separate display device.

Although the exemplary embodiments of the present invention have been described in detail with reference to the accompanying drawings, the present invention is not limited thereto and may be embodied in many different forms without departing from the technical concept of the present invention. Accordingly, the various exemplary embodiments disclosed herein are not intended to limit the technical spirit of the present invention but describe and the scope of the technical spirit of the present invention is not limited to the exemplary embodiments. Thus, it is to be appreciated that the embodiments described above are intended to be illustrative in every sense, and not restrictive. The protective scope of the present invention should be construed based on the following claims, and all the technical concepts in the equivalent scope thereof should be construed as falling within the scope of the present invention.

1. A cognitive function test server, comprising:
a communication interface;
a memory; and
a processor which is operably connected to the communication interface and the memory, wherein the processor is configured to
provide a first sequence to acquire brainwave data of a user in a resting state by means of an HMD device, acquire baseline brainwave data of the user based on the first sequence,
provide at least one second sequence related to a cognitive function by means of the HMD device,
acquire input data and activated brainwave data based on the second sequence from the HMD device and an input device connected thereto, and
generate a cognitive evaluation result of the user based on at least one of the reference brainwave data, the activated brainwave data, and the input data of the user.

2. The cognitive function test server of claim 1, wherein the processor is configured to provide the first sequence including at least one of a guide text configured to guide the user an eye-related action and an effect sound for guiding a specific time.

3. The cognitive function test server of claim 1, wherein the processor is configured to provide an image for inducing the resting state.

4. The cognitive function test server of claim 1, wherein the processor is configured to calculate a cognitive evaluation score of the user based on the input data, determine whether the user has an cognitive impairment according to the calculated evaluation score, and determine an effectiveness of the determined cognitive impairment based on a comparison result of the reference brainwave data and the activated brainwave data.

5. The cognitive function test server of claim 1, wherein the processor is configured to calculate a cognitive evaluation score of the user based on the input data, and determine whether the user has a cognitive impairment by reflecting a comparison result of the reference brainwave data and the activated brainwave data to the calculated evaluation score.

6. The cognitive function test server of claim 1, wherein the processor is configured to determine whether the user has a cognitive impairment based on a comparison result of the reference brainwave data and the reactive brainwave data, and provide a cognitive evaluation score calculated based on the input data as reference data.

7. The cognitive function test server of claim 4, wherein the comparison result is a power spectral density (PSD) comparison result for every frequency band.

8. The cognitive function test server of claim 4, wherein whether the user has a cognitive impairment is determined for every cognitive function of the user..

9. The cognitive function test server of claim 1, wherein when the processor acquires a detaching signal of the HMD device, the processor is configured to acquire the reference brainwave data of the user again by means of the HMD device.

10. The cognitive function test server of claim 1, wherein the processor is configured to measure a pupil movement of the user to compensate for a center of the pupil of the user or a distance between the centers of the pupils.

11. The cognitive function test server of claim 1, wherein the processor is configured to provide a second sequence configured to test at least one cognitive function of short-term memory, long-term memory, concentration, depth perception, quickness, visual perception, spatial perception, working memory, instantaneous attention, sustained attention, reading ability, and selective attention of the user.

12. The cognitive function test server of claim 11, wherein the second sequence configured to test the short-term memory or the long-term memory includes at least one of a plurality of graphic objects located in a three-dimensional space, a graphic object moving in the three-dimensional space, and a guide content guiding selection of and an object equal to or different form the plurality of graphic objects after a specific time, on a background of the three-dimensional space.

13. The cognitive function test server of claim 11, wherein the second sequence configured to test the concentration includes at least one of a plurality of graphic objects having a same shape, at least one graphic object exposed in a different way after a predetermined time, among the plurality of graphic objects, a motion of the plurality of graphic objects in a three-dimensional space, and a guide content guiding to select at least one graphic object exposed in the different way.

14. The cognitive function test server of claim 11, wherein the second sequence configured to test the depth perception includes at least one of a plurality of graphic objects having a same shape but located far or near from the user's point of view, and a guide content guiding to sequentially select graphic objects located far or near among the plurality of graphic objects.

15. A cognitive function testing method, comprising:
providing a first sequence configured to acquire brainwave data of a user in a resting state by means of an HMD device;
acquiring baseline brainwave data of the user based on the first sequence;
providing at least one second sequence related to a cognitive function to the user in the resting state, by means of the HMD device;
acquiring input data based on the second sequence from the HMD device and an input device connected thereto, and
generating an evaluation result for every cognitive function of the user based on the reference brainwave data and the input data of the user.
